(19) United States
(12) Patent Application Publication
(10) Pub. No.: US 2012/0173261 A1
(43) Pub. Date: Jul. 5, 2012

(54) PRESENTING AGENT ORDER SUGGESTIONS TO CLINICIANS

(75) Inventor: CHARLES SCHNEIDER, Trimble, MO (US)

(73) Assignee: CERNER INNOVATION, INC., Overland Park, KS (US)

(21) Appl. No.: 13/178,267

(22) Filed: Jul. 7, 2011

Related U.S. Application Data
(60) Provisional application No. 61/428,976, filed on Dec. 31, 2010.

(51) Int. Cl. G06Q 50/00; G06F 3/048

(52) U.S. Cl. 705/2; 715/825

(57) ABSTRACT

Computerized systems and methods are provided for automatically providing order and details suggestions to healthcare users. Healthcare users such as physicians often place the same small number of orders with the same details over and over again. A usage pattern warehouse may be employed to track and determine the user's order patterns. The usage pattern warehouse may assign a weighted score to the agent orders based upon volume and recency. User text input is matched to agent orders within the historical order information and a listing of most likely agent orders is provided to the user.

### CROSS-REFERENCE TO RELATED APPLICATIONS

This application claims the benefit of U.S. Provisional Application No. 61/428,976, filed Dec. 31, 2010. The aforementioned application is herein incorporated by reference in its entirety.

### FIELD

The present disclosure relates to orders in a healthcare system.

### BACKGROUND

Orders are communicated by members of a health care team to direct patient care activities. Typically, physicians provide orders by writing an order into a patient chart for hospitalized patients. Computerized ordering may increase efficiency and reduce health care-related errors.

### SUMMARY

Examples are directed to computerized systems and methods that may be stored on one or more computer-storage media and executable by a computing device. Clinicians often place the same small number of orders with the same details and laboriously input the same orders with the same details each time the physician places the order. The provided systems and methods reduce the keystrokes and/or clicks as well as time it takes for a physician to place an order. Generally, a user may type a few characters of the order. Orders may include medications, laboratory tests, monitoring, diagnostic tests, diet, IV lines, etc. The text input is used to search within a usage pattern warehouse, which stores user ordering patterns. The usage pattern warehouse tracks the order history of the user and assigns agent orders a weighted score based upon order volume and recency. A list of matching agent orders may be sorted according to the weighted score and displayed to the user, who may select one of these agent orders for administration. Thus, the user may quickly bring up a listing of recent and commonly used orders matching the search term without having to input the entire order. The usage pattern warehouse may also adapt to the user patterns by adding new agent order entries and tracking the evolving usage patterns.

This section provides a general summary of the disclosure, and is not a comprehensive disclosure of its full scope or all of its features. Further areas of applicability will become apparent from the description provided herein. The description and specific examples in this summary are intended for purposes of illustration only and are not intended to limit the scope of the present disclosure.

### DESCRIPTION OF THE DRAWINGS

Illustrative embodiments of the invention are described in detail below with reference to the attached drawing figures, and wherein:

FIG. 1 is a block diagram depicting an exemplary operating environment suitable for use in accordance with an embodiment of the invention;

FIG. 2 is a block diagram depicting an exemplary network architecture suitable for use in accordance with an embodiment of the invention;

FIG. 3 is a block diagram depicting a method for suggesting agent orders in accordance with an embodiment of the invention;

FIGS. 4-5 are graphical representations of an exemplary agent order suggestions in accordance with an embodiment of the invention; and

FIG. 6 is a block diagram illustrating a method for determining order usage patterns to suggest agent orders in accordance with an embodiment of the invention.

### DETAILED DESCRIPTION

The subject matter of embodiments of the invention is described with specificity herein to meet statutory requirements. But the description itself is not intended to necessarily limit the scope of claims. Rather, the claimed subject matter might be embodied in other ways to include different steps or combinations of steps similar to the ones described in this document, in conjunction with other present or future technologies. Terms should not be interpreted as implying any particular order among or between various steps herein disclosed unless and except when the order of individual steps is explicitly described.

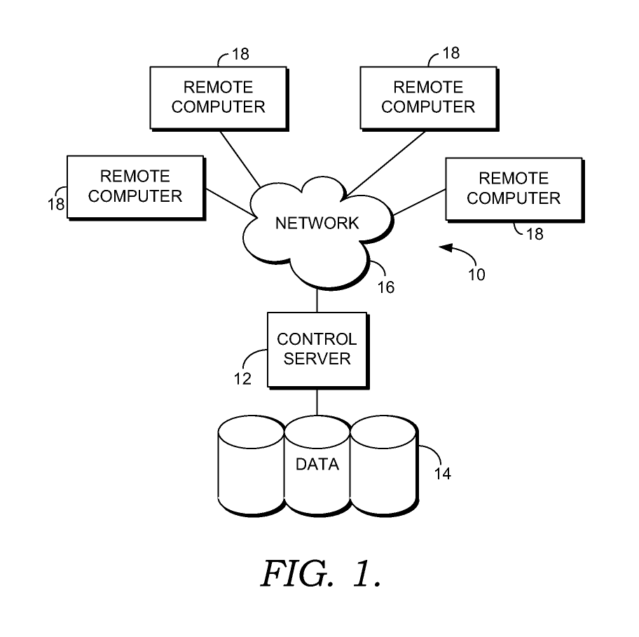

Having briefly described embodiments of the present invention, an exemplary operating environment suitable for use in implementing embodiments of the present invention is described below. Referring to the drawings in general, and initially to FIG. 1 in particular, an exemplary computing system environment, a medical information computing system environment, with which embodiments of the present invention may be implemented is illustrated and designated generally as reference numeral 20. It will be understood and appreciated by those of ordinary skill in the art that the illustrated medical information computing system environment 20 is merely an example of one suitable computing environment and is not intended to suggest any limitation as to the scope of use or functionality of the invention. Neither should the medical information computing system environment 20 be interpreted as having any dependency or requirement relating to any single component or combination of components illustrated therein.

The present invention may be operational with numerous other general purpose or special purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in association with local and/or remote computer storage media including, by way of example only, memory storage devices.

With continued reference to FIG. 1, the exemplary medical information computing system environment 20 includes a general purpose computing device in the form of a control server 22. Components of the control server 22 may include, without limitation, a processing unit, internal system memory, and a suitable system bus for coupling various system components, including database cluster 24, with the control server 22. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 22 typically includes therein, or has access to, a variety of computer-readable media, for instance, database cluster 24. Computer-readable media can be any available non-transitory media that may be accessed by server 22, and includes volatile and nonvolatile media, as well as removable and non-removable media. By way of example, and not limitation, computer-readable media may include computer storage media. Computer storage media may include, without limitation, volatile and nonvolatile media, as well as removable and non-removable media implemented in any method or technology for storage of information, such as computer-readable instructions, data structures, program modules, or other data. In this regard, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the control server 22. Combinations of any of the above also may be included within the scope of computer-readable media.

The computer storage media discussed above and illustrated in FIG. 1, including database cluster 24, provide storage of computer-readable instructions, data structures, program modules, and other data for the control server 22. The control server 22 may operate in a computer network 26 using logical connections to one or more remote computers 28. Remote computers 28 may be located at a variety of locations in a medical or research environment, for example, but not limited to, clinical laboratories (e.g., molecular diagnostic laboratories), hospitals and other inpatient settings, veterinary environments, ambulatory settings, medical billing and financial offices, hospital administration settings, home health care environments, and clinicians' offices. Clinicians may include, but are not limited to, a treating physician or physicians, specialists such as neonatologists, surgeons, radiologists, cardiologists, and oncologists, emergency medical technicians, physicians' assistants, nurse practitioners, nurses, nurses' aides, pharmacists, dieticians, microbiologists, laboratory experts, laboratory technologists, genetic counselors, researchers, veterinarians, students, and the like. The remote computers 28 may also be physically located in non-traditional medical care environments so that the entire health care community may be capable of integration on the network. The remote computers 28 may be personal computers, servers, routers, network PCs, peer devices, other common network nodes, or the like, and may include some or all of the elements described above in relation to the control server 22. The devices can be personal digital assistants or other like devices.

Exemplary computer networks 26 may include, without limitation, local area networks (LANs) and/or wide area networks (WANs). Such networking environments are commonplace in offices, enterprise-wide computer networks, intranets, and the Internet. When utilized in a WAN networking environment, the control server 22 may include a modem or other means for establishing communications over the WAN, such as the Internet. In a networked environment, program modules or portions thereof may be stored in association with the control server 22, the database cluster 24, or any of the remote computers 28. For example, and not by way of limitation, various application programs may reside on the memory associated with any one or more of the remote computers 28. It will be appreciated by those of ordinary skill in the art that the network connections shown are exemplary and other means of establishing a communications link between the computers (e.g., control server 22 and remote computers 28) may be utilized.

In operation, a clinician may enter commands and information into the control server 22 or convey the commands and information to the control server 22 via one or more of the remote computers 28 through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote healthcare device to the control server 22. In addition to a monitor, the control server 22 and/or remote computers 28 may include other peripheral output devices, such as speakers and a printer.

Although many other internal components of the control server 22 and the remote computers 28 are not shown, those of ordinary skill in the art will appreciate that such components and their interconnection are well known. Accordingly, additional details concerning the internal construction of the control server 22 and the remote computers 28 are not further disclosed herein.

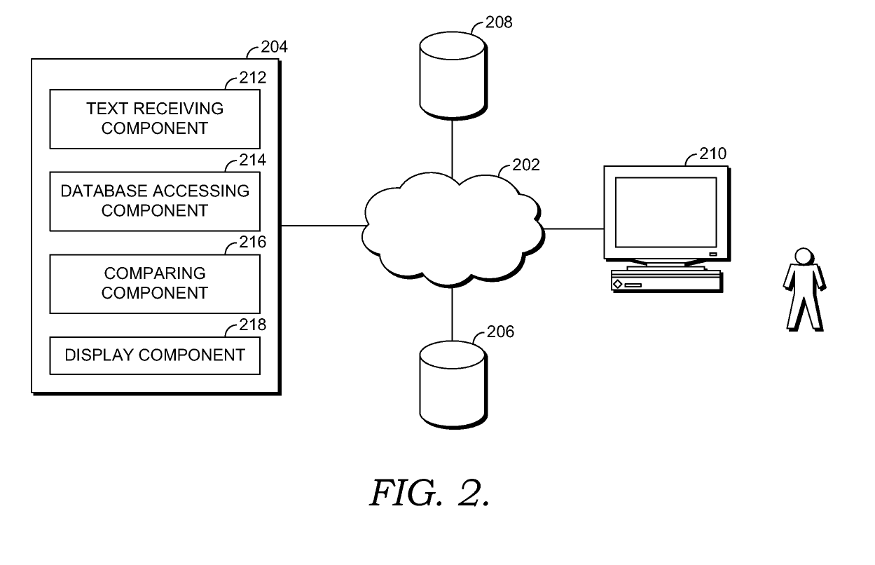

With additional reference now to FIG. 2, an exemplary network architecture 200 suitable for use in embodiments of the invention is described. The network architecture 200 may reside within or comprise a medical information computing system environment 20 described above. The network architecture 200 is one example, of which there are many, that can be used to implement embodiments of the invention. Components of the network architecture 200 are depicted singularly for clarity but, in practice, may include a plurality of similar or dissimilar components that are configured to perform the functions described below. Additionally, one or more of the components or the functions thereof can be integrated into a single component or further divided into a plurality of subcomponents. The network architecture 200 is not intended to limit components or network architectures that can be employed in embodiments of the invention. One of skill in the art will recognize other components and architectures that are suitable for use in embodiments of the invention.

The network architecture 200 includes a network 202, a order usage pattern manager 204, an Electronic Medical Record (EMR) database 206, a historical agent information database 208 and a user's computing device 210. The network 202 includes any available network, such as for example, an intranet, the Internet, an Ethernet, a local area network, and the like as described above. In an embodiment, the network 202 is a secure local area network of a healthcare system such as a hospital.

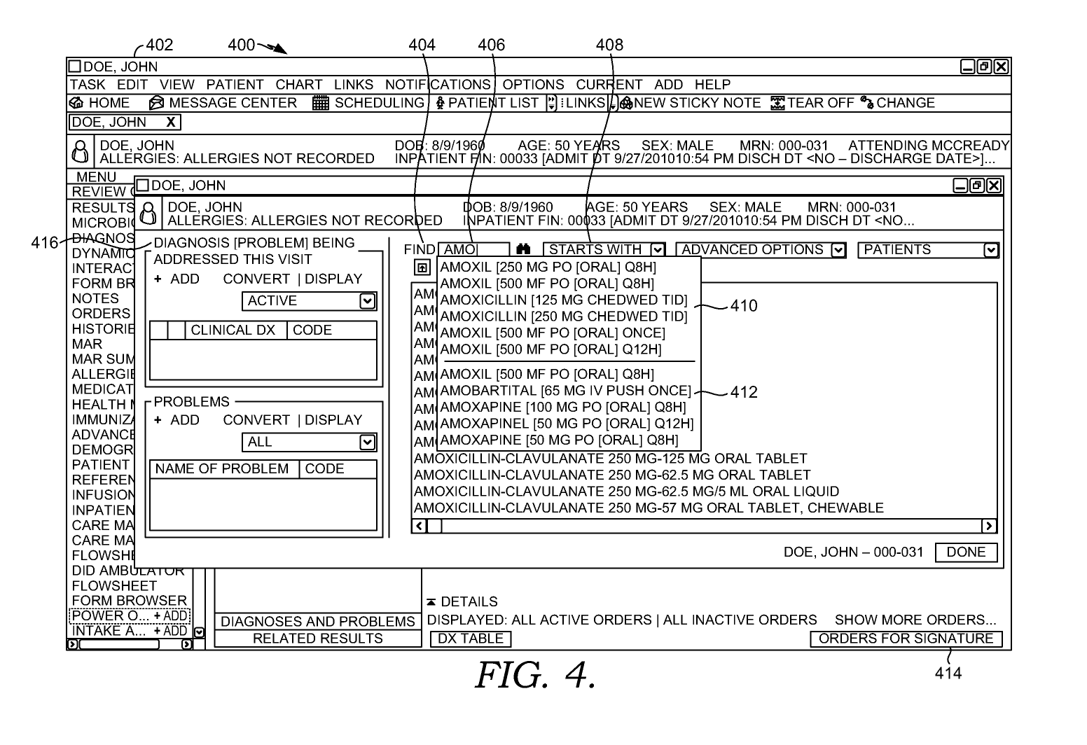

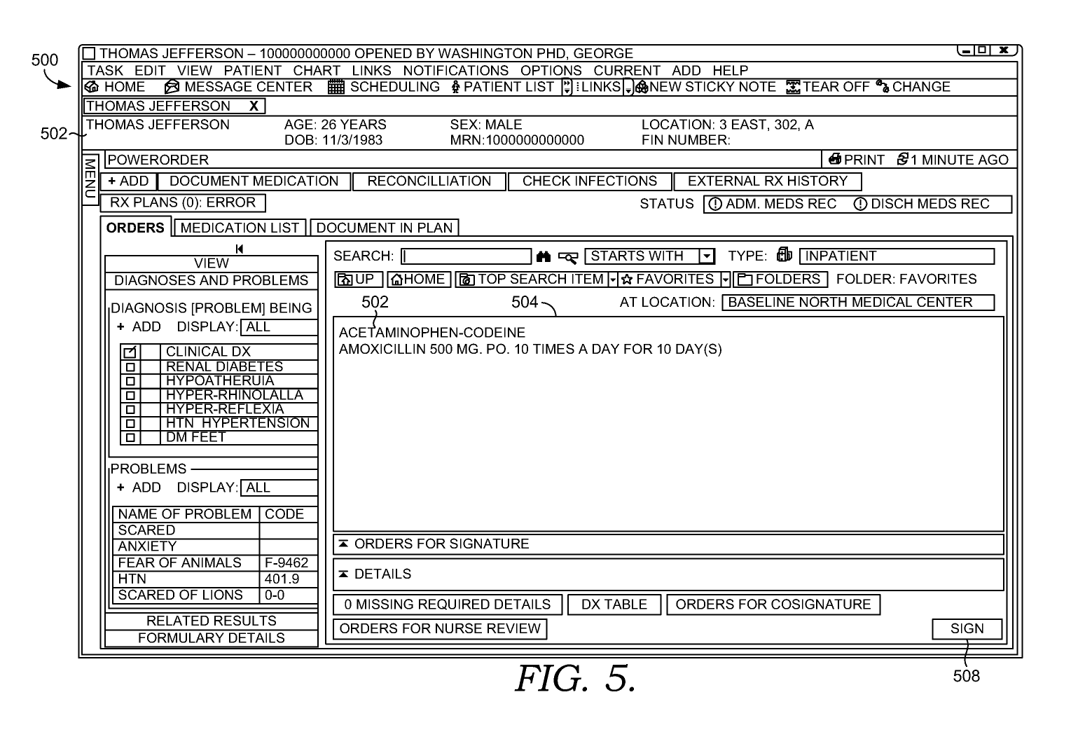

With continued reference to FIG. 2, the network architecture 200 also includes the user's computing device 210. The user's computing device is any available computing device, such as the control server 22 or the remote computers 28 of FIG. 1. In an embodiment, the user's computing device 210 and the network computing device 204 are the same computing device. The user's computing device 210 is communicatively coupled to the network 202 and thereby to the order usage pattern manager 204, the EMR database 206 and the historical agent information database 208. The user's computing device 210 includes an associated display device and is operated by a user or clinician. The display device is any display device available in the art suitable for providing a display to the clinician of a graphical user interface, as described more fully below with reference to FIGS. 4 and 5.

The user's computing device 210 may be employed by the clinician to access and interact with an EMR for a patient. An EMR is an electronic version of a patient's medical record or chart as is known in the art. The EMR presents patient data for a respective patient that is stored in the EMR database 206 and allows clinicians to add, input, alter, access, or otherwise interact with the patient data. The EMR is provided by any available applications and in any desired format known in the art. In an embodiment, the EMR and other graphical user interfaces are presented in a web page-style format and include an initial page or portal that is presented to the clinician upon accessing the EMR, such as depicted in FIG. 4. Such a presentation may employ hypertext markup language (HTML), Java script, or any other available coding.

The order usage pattern manager 204 is executed on one or more computer computing devices, such as control server 22, and is functional to suggest orders to a clinician. Orders may include any physician instructions to other healthcare providers. Orders may be for medications, laboratory tests, diagnostic test, fluids, consultations, activity, monitoring, and/or diet. Agents may be order specifics: for example, an EKG is an agent of diagnostic test orders. Agents may also include other parameters or details of an order such as priority, reason for exam, dispense as written, mode of transport, etc. Examples of medications or prescription drug order suggestions include, a prescription order sentence that includes, the name of a drug to be prescribed, dose, route of administration and frequency of administration to be prescribed. Exemplary agents include pulse oximetry for monitoring, Foley catheter for tubes, MRI for diagnostic tests, social service consults, etc.

The order usage pattern manager 204 includes a number of components. While specific components and devices are illustrated and discussed hereinafter, it is understood that additional or fewer components may be employed as part of the order usage pattern manager in various embodiments of the present invention. As illustrated, the order usage pattern manager includes a text receiving component 212, a database accessing component 214, a comparing component 216 and displaying component 218.

The receiving component 212 receives component text input by a clinician. In an exemplary embodiment a healthcare provider, a manufacturer, or provider may input text into a text box displayed on a clinician device 210 by display component 218. Typically, the text entered by a clinician includes alphanumeric information for the name or portion of the name of an agent. For example, text for “amo” may be entered by a clinician via text box displayed on clinician device 210, all of which will be discussed in greater detail with reference to FIG. 4.

The database accessing component 214, accesses historical agent information database 208. The historical agent information database 208, in an exemplary embodiment, stores information regarding previous agent orders made by a particular clinician, clinicians at an organization, clinicians in a geographic region, a group of clinicians and/or any combined pool of these. The historical information regarding previous agent orders made is stored and then is utilized by the comparing component 216 to determine a suggested order for an agent upon the receiving component 202 receiving a text input. Therefore, while the historical data from historical agent information database 208 may be very specialized to a particular requester (e.g., physician), it may draw on the experience of a greater group such as a whole organization (e.g., group of hospitals). Additionally, the historical information may be anonymous or otherwise blind to maintain information privacy.

In one example, the historical agent information database 208, may comprise a usage pattern warehouse. The usage pattern warehouse may receive orders with details and store them in a table. The usage pattern warehouse may determine whether or not an order placed by a user has been placed previously with the same details by this user. For example, a user places an order, which may be received by the order usage pattern manager. If the agent order has not been placed previously by the user and/or cannot be found within the historical agent information database 208, a new entry for the agent order is added to the warehouse for that user. If a match is found, then the volume (i.e. the number of times this user has placed this order with these details) is updated by incrementing. In addition, the recency (i.e. how recently this user has placed this order) may be updated. A weighted score may be calculated for the agent order based upon the volume and recency of the order.

To illustrate, a user places an order for “Left Arm X-Ray Stat.” If the user places the “Left Arm X-Ray Stat” many times a day and continues to do so, the volume and the recency are both very high. Thus the “Left Arm X-Ray Stat” agent order will have a high weighted score. If the user places the “Left Arm X-Ray Stat” agent order once a month, the volume and recency may be lower. If the user placed the “Left Arm X-Ray Stat” agent order frequently last year, but this year the user has only placed the “Left Arm X-Ray Stat” agent order a few times, the weighted score for the agent order may drop from last year's score. In another example, if a user has occasion to place the “Left Arm X-Ray Stat” many times in one day, the recency may cause the weighted score for the agent order to increase for that day. As a result, the usage pattern warehouse allows the displayed orders to adapt to the current needs of the user.

Moreover, the usage pattern warehouse may keep track of the user's patterns by department or service. For example, an internist may spend part of her time at a larger city hospital and part of her time working in a rural setting. The agent orders placed by the internist may exhibit different usage patterns due to the different demographics of patients and situations encounters. Another example may include residents who often rotate through services: a resident may be on transplant for a period and then change to trauma, in which case the order usage pattern for the resident would differ greatly between the two services. Thus the usage pattern warehouse may categorize or track the service, department, location, or the like for the user in order to provide relevant usage patterns.

The comparing component compares the text received by receiving component 202 to the historical agent information database 208 comprising the usage pattern warehouse. For example, if a user types in “left” into the search text box, the comparing component compares this text to the usage pattern warehouse information for “left” matches. If twenty entries are found, the weighted score for the entries is used to determine which of the 10 highest scored agent orders will be returned. If the user wishes to return all matches, the matching list may be stored based upon the weighted scores. This list may be displayed to the user as described below and includes agent orders that the user places the “most” and most likely wants to place again.

For example, referring to FIG. 4, a text entry of “amo” entered by Dr. X in a text box 404, is compared with historical agent order information for Dr. X who is currently logged onto the system. The comparing component 216 of the order usage pattern manager 204 determines the most frequentagent orders 411 of Dr. X beginning 408 with the letters “amo” 406. With reference to FIG. 4, the comparing component 216 determines that most frequently prescribed order sentences beginning with the letters “amo” for Dr. X are AMOXIL [250 MG PO [ORAL] Q8H] and AMOXIL [500 MF PO [ORAL] Q8H] and so on. The most frequentorders 411 for Dr. X beginning with the letters “amo” are displayed by the displaying component 218 in the drop down menu 410.

The comparing component 216 also determines the most frequent agent orders for all clinicians in Dr. X's facility beginning with the letters “amo” and the most frequent orders for all clinicians 412 are displayed by displaying component 218 in drop down menu 410. The display of suggested orders allows for the clinician to easily see his or her most frequent orders and those of other clinicians within the organization. The frequency, route and dosage information along with the name of the agent are included in the drop down menu 410.

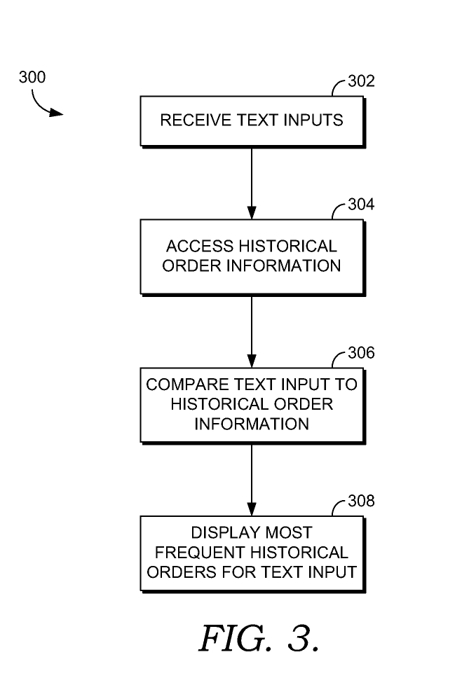

Displaying component 218 displays the suggested agent orders in drop down menus and graphical user interfaces are presented in a web page-style format and includes an initial page or portal that is presented to the clinician upon accessing the EMR, such as depicted in FIG. 4. Such a presentation may employ hypertext markup language (HTML), Java script, or any other available coding. Several configurations may be available for the list of suggest agent orders. For example, the user may choose to display a list of “My Top 100” or “My Top X,” wherein “X” is a number of displayed entries set by the user. The list of suggested agent orders will then display the 100 (or 50 or whatever number the user chooses) agent orders that have the highest weighted score. The user may select one of these high scoring agent orders with or without inputting a search term. In another example, user may input a portion of the agent order name into a search text box. The user text entry may be used to search within the usage pattern warehouse for matches. Depending on the number of matching entries, the usage pattern warehouse may return all or a portion of these entries. For example, a search for the “Left” term may return 100 matches but only 10 score above a certain threshold based on volume and recency. These 10 entries may be presented to the user as the matching orders he/she place most often and most likely would place again. In addition, if these matches are unsuitable, the user may select an option to retrieve more matches or display all matches or input additional search terms. Turning to FIG. 3, a flow diagram showing a method 300 performed by one or more computing devices for displaying suggested agent orders is described. Initially, at block 302 alphanumeric text inputs are received from a user. At block 304, historical agent order information for the user and/or a group of users is accessed. As described previously, the historical agent order information may be used to determine user order patterns via the usage pattern warehouse. Based on the user text input, a match is sought within the historical agent order information. At block 306, the text inputs are compared with the historical agent order information in order to determine the most frequent historical orders for the text input. The most frequent historical orders may be determined by the usage pattern warehouse. Each time an agent order is selected by the user, the volume for the agent order may be incremented and the recency tracked. These two parameters may contribute to a weighted score. This weighted score may be used to assess the most frequent historical orders. The weighted score may be determined by the order volumes and recency over a group of users. The most frequent historical orders may also be determined by user parameters such as practice location or service. At block 308, the most frequent historical agent orders for the text input are displayed in a graphical user interface for the user to easily select and place for the order for a patient.

Turning to FIG. 4, graphical user interfaces (GUI) 400 provides a text input box 404 allowing a user to input text related to an agent order to be placed for a patient 402. The user can enter search text 404 into textbox 406. The user can specify whether the suggested agent order they are searching for starts with, contains, ends with or any other variation the input text into field 408. After the agent suggestion manager searches and compares the input text with historical agent orders, the suggested agent orders are displayed in a drop down menu 410 for the requesting users 411 and/or for a group of users 412. In addition, the user may input into field 416 information regarding the patient's condition.

Upon selection by a user or clinician of a suggested agent order from drop down menu, the user my select the field 414 to sign the suggested agent order so that it is signed and placed within a computerized medical ordering system. The selected agent order then becomes an actual order for patient 402 within the system to be completed for the patient.

Referring next to FIG. 5, graphical user interfaces (GUI) 500 provides within the context of a patient 502, a listing the most frequently placed orders 504 by the clinician logged onto the system. Any number of previously placed agent orders 506 may be listed in field 504. In addition, the clinician may select from the list of previously placed agent orders to place the order for patient 502. Upon selection by a user or clinician of a suggested agent order from drop down menu, the user my select the field 508 to sign the suggested agent order so that it is signed and placed within a computerized medical ordering system. The selected agent order then becomes an actual order for patient 502 within the system to be completed for the patient.

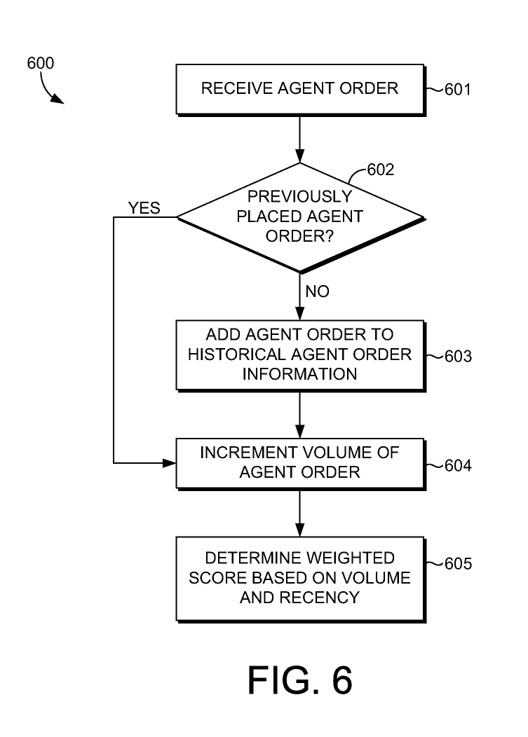

Referring to FIG. 6, a flow diagram illustrates a method 600 performed by one or more computing devices for collecting historical agent order information to provide order and details suggestions to healthcare users. At block 601, an agent order may be received. At block 602, the comparing component determines if the historical agent information database and/or usage pattern warehouse has one or more matches for previously placed agent orders. If there is no match, the new agent order may be added to the historical agent order information at block 603. If the agent order has been previously ordered by the user, the volume for the agent order may be incremented and the recency updated at block 604. The weighted score for the agent order may be determined based on the volume and the recency at block 605. This weighted score may be stored in association with the historical agent order information

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

The invention claimed is:

1. One or more computer storage media storing computer-useable instructions that, when executed by one or more computing devices, cause the one or more computing devices to perform a method comprising:
receiving an agent order for a patient from a user;
determining, from historical agent order information for the user, if the user has previously ordered the same agent order;
if the user has previously ordered the same agent order, incrementing a volume of a total number of times the user has ordered the agent;
determining a weighted score for the agent order based on a volume and a recency of the agent order;
storing the count and the weighted score with the historical agent order information.

2. The method of claim 1, wherein receiving an agent order for a patient from a user comprises:
receiving a user text entry of a portion of a name of the agent order for the patient;
accessing the historical agent order information for the user;
comparing the user text entry to the historical agent order information for the user to determine potential agent orders of the user based on the user text entry;
displaying to the user a list of previously placed agent orders based on weighted scores as suggested agent orders that may be selected by the user to be placed for a patient; and
receiving an order for the agent order by the user.

3. The method of claim 2, wherein the list of previously placed agent orders is further determined by a service associated with the user.

4. The method of claim 2, wherein the list of previously placed agent orders is further determined by manual input received from the user.

5. The method of claim 2, the method further comprising:
if the suggested agent orders do not include the agent order requested by the user, adding the agent order to the historical agent order information.

6. The method of claim 2, wherein agent orders comprise laboratory tests, diagnostic tests, medications, fluids, consultations, activity, monitoring, and diet.

7. A computerized system for suggesting agent orders to a user in a healthcare environment, the system comprising:
an order usage pattern manager for receiving a user text entry of a portion of a name of an agent to be ordered for a patient and further for comparing the user text entry to historical agent order information for the user; and
a usage pattern warehouse, accessible by the order usage pattern manager, configured to store and analyze historical agent order information for user usage patterns.

8. The system of claim 7, the usage pattern warehouse further configured to determine a ranking of agent orders by at least one of frequency or recency.

9. The system of claim 7, the order usage pattern manager further configured to determine and display suggested agent orders that may be selected by the user to be placed for a patient.

10. The system of claim 7, the usage pattern warehouse further configured to sort historical agent order information by a department rotation of the user.

11. The system of claim 9, the order usage pattern manager further configured to receive a selection of at least one suggested agent order by the user and transmitting the selection to the usage pattern warehouse.

12. The system of claim 9, the order usage pattern manager further configured to receive a user input of at least one agent order not listed in the suggested agent order and transmitting the user input to the usage pattern warehouse.

13. The system of claim 9, the order usage pattern manager further receiving the user input configuring the suggested agent order display.

14. A graphical user interface (GUI) stored on one or more computer-storage media and executable by a computing device, said GUI comprising:
a text entry display area for the input of text by user of a portion of a name of an agent to be ordered for a patient;
a display menu area including a list of the most frequent agent orders for the user; and
a order signature area for the user to sign and order one or more of the most frequent agent orders displayed in the drop down display menu area.

15. The GUI of claim 14, further comprising: a new agent order area for the user to input at least one agent order not displayed in the list of the most frequent agent orders.

16. The GUI of claim 14, the list of most frequent agent orders for the user is determined by volume of agent orders for the user.

17. The GUI of claim 14, the list of most frequent agent orders for the user comprising the most frequent agent orders for the user based on the text entered in text entry display area.

18. The GUI of claim 14, wherein agent orders comprise laboratory tests, diagnostic tests, medications, fluids, consultations, activity, monitoring, and diet.

19. The GUI of claim 14, the list of most frequent agent orders for the user is determined by volume of agent orders for the user and by an additional user field.

20. The GUI of claim 19, wherein the additional user field is a department of hospital associated with the user.
